(19) United States
(12) Patent Application Publication
(10) Pub. No.: US 2019/0209728 A1
(43) Pub. Date: Jul. 11, 2019

(54) Anti-Bacterial Air Filtering Apparatus

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 15/866,387

(22) Filed: Jan. 9, 2018

(51) Int. Cl. A61L 9/20; B01D 53/04; B01D 46/00; B01J 20/20; B01J 20/28; B01J 21/06; B01J 35/00; A41D 13/11; A62B 23/02

(52) U.S. Cl. CPC A61L 9/205; A61L 2209/22; B01D 46/0032; B01D 46/0028; B01J 20/20; B01J 20/28023; B01J 21/063; B01J 35/004; A41D 13/1192; A62B 23/025; A41D 13/1161; B01D 2253/102; A61L 2209/12; A61L 2209/14; B01D 53/04

(57) ABSTRACT

An air filtering apparatus using at least three layers of air-permeable mesh structure is disclosed where at least one layer of air-filtering mesh structure contains an anti-bacterial composite material, and at least one layer of air-permeable mesh structure contains material for absorbing airborne non-microbial particles. The combination of three-layer air-permeable mesh structure is effective against airborne microbial matters and volatile organic compounds. A face mask employing such 3-layer air-permeable fabric is also introduced and can be used for effective protection for a prolonged period of time.

### BACKGROUND

### Technical Field

The present disclosure pertains to the field of air filtering devices and, more specifically, proposes an anti-bacterial air filtering device.

### Description of Related Art

Photocatalysts are known to become active under ultraviolet light and kill bacteria by breaking down the cell wall of the bacteria. Day, E., et al., in U.S. Pat. No. 8,709,341 teaches the use of a photocatalytic element made of anatase-type titanium oxide (TiO₂) in an air filtering device such that when the UV light shines on the photocatalytic element, it activates the photocatalytic element, causing it to break down the bacteria cell wall and resulting in the killing of the bacteria. One main limitation of this anti-bacterial air filtering device is the need for a UV core. Without the UV core, the photocatalytic element will not be activated. Because it is harmful to expose users under UV light for extended periods of time, the UV core in the teaching of Day, E., et al., needs to be sealed, and this constitutes another design limitation.

Another issue that necessitates a closer inspection of the lighting device by Day, E., et al. is the effectiveness of the anatase-type titanium oxide. In U.S. Pat. No. 9,522,384, Liu L. et al. explained that the particles of anatase-type titanium oxide may be of two shapes: sphere and rhombus. The sphere-shape particle is larger, whereas the rhombus-shape particle is smaller, meaning that the latter has a higher density per unit of volume and therefore a much higher photocatalytic bacteria-killing effect than that of the former. This means that for an anatase-type titanium oxide film of a given thickness, it would take much less UV ray spectral power for the rhombus-shape particle to generate the same amount of germicidal effect as compared to the amount of UV ray spectral power required by the sphere-shape particles. Day, E., et al., in U.S. Pat. No. 8,709,341 did not address the difference in the photocatalytic effectiveness of different particle shapes of the anatase-type titanium oxide, nor the differing amounts of UV light spectral power as needed by the different particles shapes of the anatase-type titanium oxide for generating the same photocatalytic germicidal effect.

In FIG. 1, the spectral power distribution (SPD) of different light sources are shown. From the SPC curves, it can be observed that daylight contains significant UV ray power (for rays with wavelength less than 400 nm), whereas artificial light sources have significantly less UV ray power. Most notably, the LED light source exhibits nearly no UV ray power. When taking the UV ray power of different light sources into consideration, it can be argued that for anatase titanium oxide film of a given thickness, the photocatalytic germicidal effect would differ depending on the light source. In other words, in order to produce the desirable photocatalytic germicidal effect on a given thickness of the anatase titanium oxide film, the issue lies not in whether the light that shines through the film contains UV rays or not, but rather in the UV SPD of the light source. Day, E., et al., in U.S. Pat. No. 8,709,341 did not address the UV SPD requirement for the light source.

The present disclosure presents an anti-bacterial air filtering apparatus that makes use of a multi-layer air-permeable mesh structure such that each layer performs a different air filtering function and at least one layer employs the new photocatalytic composite material invented by Liu, L. et al. With this new invention, the need for a sealed UV core for germicidal irradiation can thus be eliminated. Various embodiments of the present disclosure, some with a light source and others without, are also discussed.

### SUMMARY

In one aspect, the anti-bacterial air filtering apparatus comprises a housing with at least one air inflow port and at least one air outflow port, at least one forced air mechanism, and at least one air-permeable subsystem. The forced air mechanism, disposed inside or partially inside, or outside of the housing, forces the unfiltered air to flow into the apparatus through the air inflow port, through the housing, and the filtered air out of the air outflow port. A fan inside of the housing of the present disclosure qualifies as a forced air mechanism. Placing the present disclosure inside the airway of a HVAC system, thus leveraging the forced airflow of the HVAC system (or the air circulation system in an automobile) is also considered an embodiment of the present disclosure since the housing of the airway would effectively be the housing of the present disclosure, the forced airflow of the HVAC system would be the external forced air mechanism as required by the present disclosure, and the upstream portion of the airway is regarded as the air inflow port and the downstream port of the airway is regarded as the air outflow port of the present disclosure.

Moreover, the air-permeable subsystem is disposed in the airway between the air inflow port and the air outflow port inside the house. The air-permeable subsystem comprises at least three layers of air-filtering mesh structure. At least one layer of air-filtering mesh structure contains an anti-bacterial composite material comprised of photocatalytic particles and nano silver particles for filtering microbial matters, and at least one layer of the air-filtering mesh structure contains material for absorbing airborne non-microbial particles.

Silver ions are known of having an antimicrobial effect and are not affected by light, since they are not a photocatalytic material. Using an anti-bacterial composite of photocatalytic particles and nano silver particles on at least one air-filtering mesh structure layer of the air-permeable subsystem of the air-filtering system can thus provide the bacterial killing function with or without UV light, thus overcoming the limitation of the teaching of Day, E., et al. of having a UV core on at all times. However, this anti-bacterial killing layer of air-permeable mesh structure is not effective in removing airborne non-microbial matters, such as volatile organic compounds (VOC). To improve its effectiveness for general air-filtering applications, the present disclosure makes use of at least one other layer of air-permeable mesh structure for absorbing airborne non-microbial particles.

The effectiveness of both the anti-bacterial layer and the non-microbial particle-absorbing layer depends on the underlying materials that come into physical contact with airborne matter. If the surface areas of these layers are covered with dust, then their intended usefulness is greatly reduced. As such, the proposed air-permeable subsystem makes use of a third layer in addition to and in front of the non-microbial particle absorbing layer and the anti-bacterial layer so as to prolong the effectiveness of these two layers. The tri-layer air-permeable mesh structure is one key feature of the present disclosure.

With some embodiments, two layers of air-permeable mesh structure contain an anti-bacterial composite material comprised of photocatalytic particles and nano silver particles for filtering microbial matters; one layer of air-permeable mesh structure contains material for absorbing airborne non-microbial particles; and the two anti-bacterial layers sandwich the non-microbial particle absorption layer in the middle.

With some embodiments, the photocatalytic particle contained in at least one anti-bacterial layer is rhombus-shape anatase-type titanium oxide (TiO₂). This is meant to utilize the high volume density of this type of titanium oxide, and the subsequently higher effectiveness in photocatalytic bacterial killing.

With some embodiments, the material contained in at least one chemical-compound absorbing layer is activated carbon, which is a material known for removing airborne VOCs.

With some other embodiments, the material contained in at least one chemical-compound absorbing layer is electrostatic filter fabric for it is effectiveness in removing small airborne matter such as PM 2.5.

When sufficient light passes through the air inflow port, or the air outflow ports, or both, to reach the photocatalytic material in the air-permeable subsystem, the photocatalytic material will be active in killing bacteria. However, with some embodiments, there may not be sufficient light passing through either the air inflow port or the air outflow port to activate the photocatalytic material. As such, with some embodiments, a part of the housing may be transparent or translucent in order to provide sufficient light for activating the photocatalytic material.

With some embodiments, there is at least one light source disposed inside the housing. This is to ensure the activation of the bacterial-killing photocatalytic material even when the present disclosure is placed in a sealed environment and does not receive light from any external light source. The light source used in these embodiments is not restricted to UV light source as taught by Day, E., et al. With the present disclosure, any light source where the spectral power of its UV components is between 0-5% of the total spectral power of the light source may be used.

According to FIG. 1, the spectral power of UV component of daylight is between 5-10% of the total spectral power of daylight. For most artificial light sources, the spectral power of their UV components is between 0-5% of the total spectral power of the light sources. To verify the anti-bacterial effectiveness of using an artificial light with a spectral power of UV components of less than 5% of the total spectral power of the light source, the following test was conducted. An untreated nonwoven fabric and another nonwoven fabric coated with an anti-bacterial composite film of rhombus-shape anatase-type titanium oxide (TiO₂) particles and nano silver particles were tested according to the AATCC 100 Test for Antimicrobial Test Method For Textile/Fabrics (http://www.accugenlabs.com/aatcc%20100.html).

The test was conducted over a 24-hour period in a normal test lab setting, illuminated by typical linear troffer luminaires with linear fluorescent tubular lamps set at a 12-hour-on and 12-hour-off operation cycle. The specimen fabric was placed on the lab table, at an estimated distance of 10 feet from the linear troffer luminaires on the ceiling. The results are shown in Tables 1 and 2 in FIG. 3, and some relevant remarks are listed below:
Bacteria counts should be at 1.0×10⁵ to 2.0×10⁵ CFU/m at 0 hr.
Reduction (R) %=100*(Control test at 0 hr−Treated test after 24 hr)/Control test at 0 hr.

The treated sample (with the anti-bacterial composite coating) shows a Reduction (R)=99.9% for Staphylococcus Aureus bacteria and a Reduction (R)>99.9% for Escherichia Coli bacteria. The test was conducted under a regular fluorescent light source, where the spectral power of its UV components is less than 5% of the total spectral power of the light source. It is noticeable that for untreated nonwoven fabric, the bacteria counts actually increased over 24 hours (240 folds for Staphylococcus Aureus and over 6800 folds for Escherichia Coli). The test demonstrates the bacteria-killing effectiveness of the composite film of rhombus-shape anatase-type titanium oxide (TiO₂) particles and nano silver particles exposed to a fluorescent light source with a 50% on-cycle.

Among commonly seen artificial light sources, LED exhibits the lowest percentage of UV spectral power. However, the UV spectral power of white LED for general lighting is not zero, unless it is a special made UV-free LED. An enlarged view of the SPD of a typical white LED is shown in FIG. 2. A closer look at the SPD curve reveals there are residual UV rays below 400 nm. Even though the percentage of these residual UV rays may be less than 1% of the total spectral power of the light emitted by the LED, their presence could nonetheless activate the photocatalytic particles contained in the anti-bacterial composite material on an air-permeable mesh structure. White LED may thus leverage the anti-bacterial effect of the rhombus-shape anatase-type titanium oxide.

Is it practical to use a LED light source with an anti-bacterial composite contained in an air-permeable fabric for the intended anti-bacterial function, given its small percentage of UV spectral power? The answer is three-fold. Firstly, the nano silver particles are active in killing bacteria at all times, regardless of the presence of any light or the percentage of the UV component of the light source. Secondly, the light intensity is inversely proportional to the square of the distance between the air-permeable fabric and light source (1/R2, where R is the distance from the light source). For the sake of illustration, we assume that the average distance of the LED light source disposed inside the housing of the present disclosure is 6″ from the air-permeable layer containing the photocatalytic composite material. The UV spectral power of the LED light source may be less than 1% of the total spectral power. However, the 6″ spacing between the photocatalytic layer and the LED source is 1/20 of the estimated distance (10 ft) between the treated nonwoven fabric sample and the linear fluorescent troffers of the stated AATCC 100 Test above. Thus, an air-permeable fabric positioned at 6″ from the light source will receive 20×20=400 times the light intensity of an air-permeable fabric positioned 10 ft away from the light source, implying a proportionally stronger UV spectral power at 6″ than at 10 ft. Thirdly, the rhombus-shape anatase-type titanium oxide particles are highly active when exposed to UV light, and the density of the TiO₂ particles counts more than the percentage of the UV spectral power because there is a continuous, unlimited supply of UV rays when the LED light source is turned on. When all of these factors are considered, it is practical to use a LED light source inside the housing of the present disclosure to activate the anti-bacterial composite film of rhombus-shape anatase-type titanium oxide particles and nano silver particles for achieving effective germicidal irradiation. Therefore, in some embodiments of the present disclosure, the at least one light source is made of LED (light emitting diode).

With some embodiments, the light source disposed inside the housing may be powered by external electricity. With some other embodiments, the light source may be powered by battery. With its low power consumption, a battery-power LED light source enables the use of the present disclosure with active bacteria-killing in an otherwise lightless environment for a long period of time.

In another aspect, the anti-bacterial face mask device comprises at least three layers of air-permeable mesh structure wherein two layers of air-permeable mesh structure contain an anti-bacterial composite material comprised of photocatalytic particles and nano silver particles for filtering microbial matters, and one layer of air-permeable mesh structure containing material for absorbing airborne non-microbial particles, and the two anti-bacterial layers sandwich the non-microbial particle absorption layer in the middle. The need for two anti-bacterial layers becomes apparent in the case of a face mask, since the airflow in this case is bi-directional. While the outside anti-bacterial layer prevents external airborne bacteria from getting through the face mask, the inside anti-bacterial layer prevents the bacteria exhaled by the wearer from escaping and being transmitted to others. Moreover, the inside anti-bacterial layer would kill the bacteria exhaled by the wearer, instead of simply allowing the bacteria to accumulate on the surface of the mask, thus eliminating the possibility of self-contraction.

Conventional face masks only filter the airborne bacteria, but do not kill the bacteria. As such, over a short period of time, the bacteria begins to accumulate on the small surface of a face mask. Referring to the data in Tables 1 and 2, the bacteria can grow exponentially when left untreated. Thus, a face mask without an anti-bacterial function becomes a perfect environment for bacteria growth, and the warm air breathed out by the face mask wearer further facilitates the bacteria growth. Bacteria can quickly fill the pores of face mask and spill over, thus turning the face mask into a bacterial transmitting media. This is why reusing face masks is not recommended, and healthcare professionals normally limit each use of a single face mask to 2 hours. With the present disclosure, the bacteria that come into contact with the anti-bacteria layers are killed, thus there is no proliferation of bacteria. The newly devised face mask can be safely used for a prolonged period of time, e.g., a week, with no reduction in its bacterial killing effect.

With some embodiments, the photocatalytic particle used in the anti-bacterial layer of the face mask is rhombus-shape anatase-type titanium oxide (TiO₂). With its high volume density and non-toxic nature, the rhombus-shape anatase-type titanium oxide is a preferred photocatalytic material for face masks.

With some embodiments, the material contained in at least one chemical-compound absorbing layer is activated carbon, which is a material known for removing airborne VOCs.

With some other embodiments, the material contained in at least one chemical-compound absorbing layer is electrostatic filter fabric for it is effectiveness in removing small airborne matter such as PM 2.5.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 shows the spectral power distribution (SPD) of different light sources.

FIG. 2 shows an enlarged view of the SPD of a typical white LED.

FIG. 3 contains tables showing experimental results.

FIG. 4 schematically depicts a diagram of a portable air-filtering machine wherein a fan in the housing forces the air to flow from the air inflow port through the 3-layer air-permeable filter to the air outflow port, and the two outside layers of the 3-layer air filter are coated with anti-bacterial composite film and the middle layer contains activated carbon.

FIG. 5 schematically depicts a diagram of a portable air-filtering machine similar to that of FIG. 4, except a light source is disposed inside the housing and continuously activates the photocatalytic material in the anti-bacterial composite film.

FIG. 6 schematically depicts a diagram of a replaceable air-filter insert with a battery-powered LED strip for use inside the airway of a HVAC system, and it has a 3-layer air-permeable filter where two outer layers are coated with anti-bacterial composite film and the middle layer contains activated carbon.

FIG. 7 schematically depicts a diagram of a face mask with a 3-layer air-permeable filter, where the two outer layers are coated with anti-bacterial composite film and the middle layer contains electrostatic filter fabric.

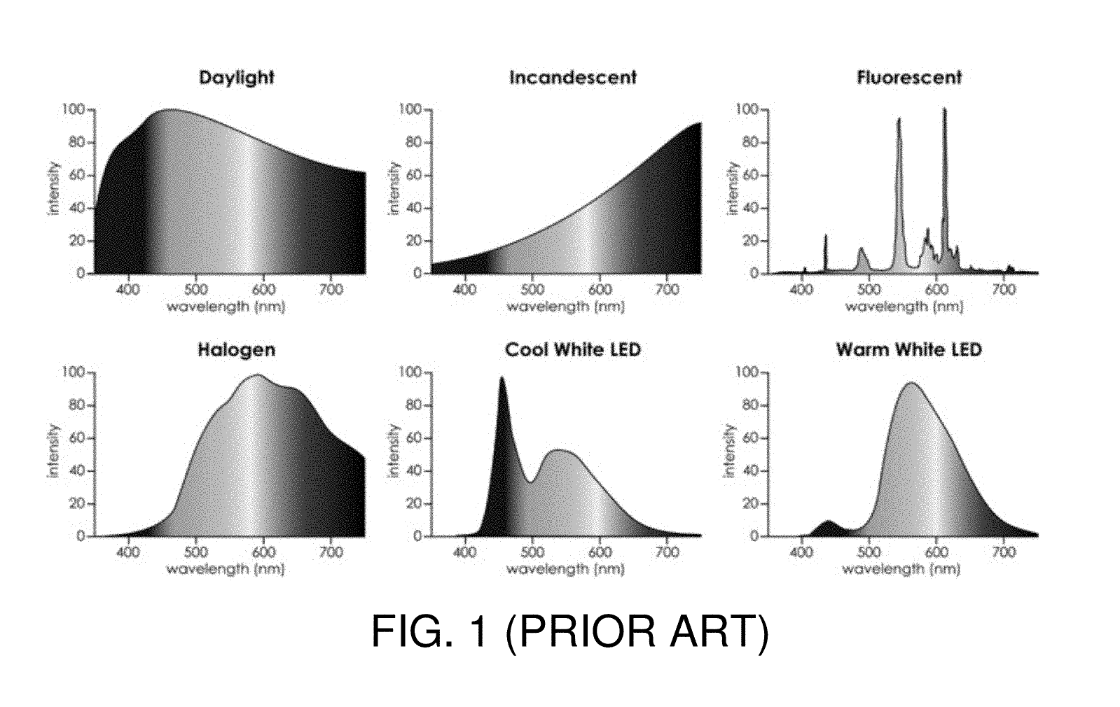

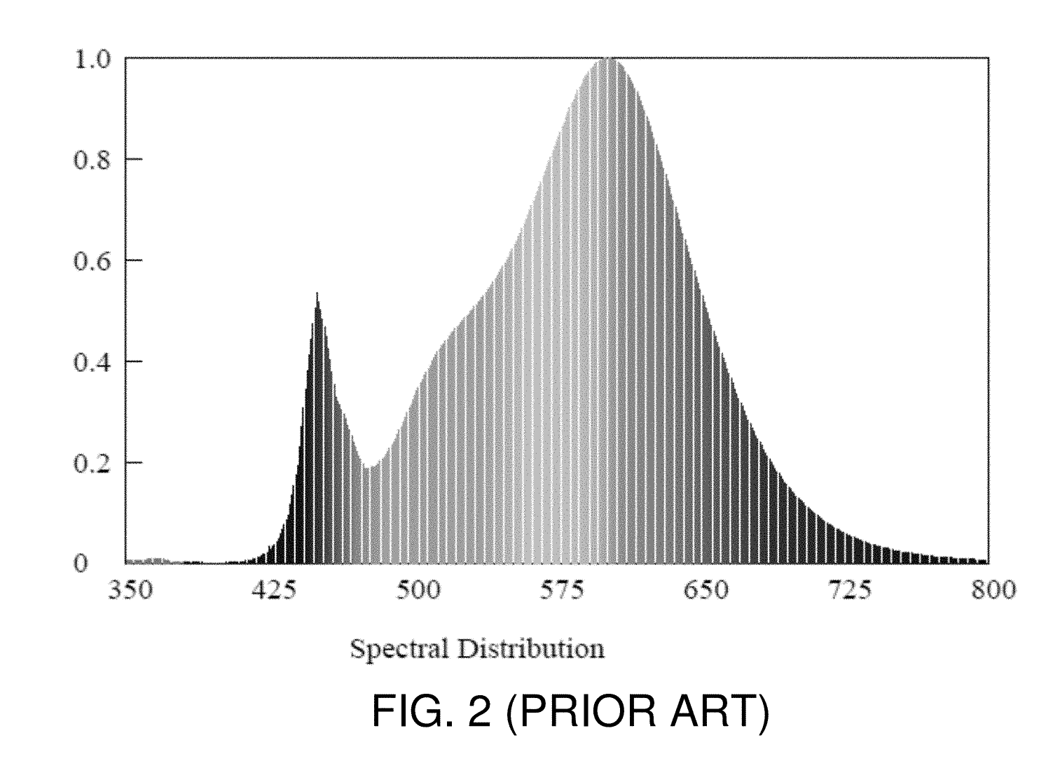

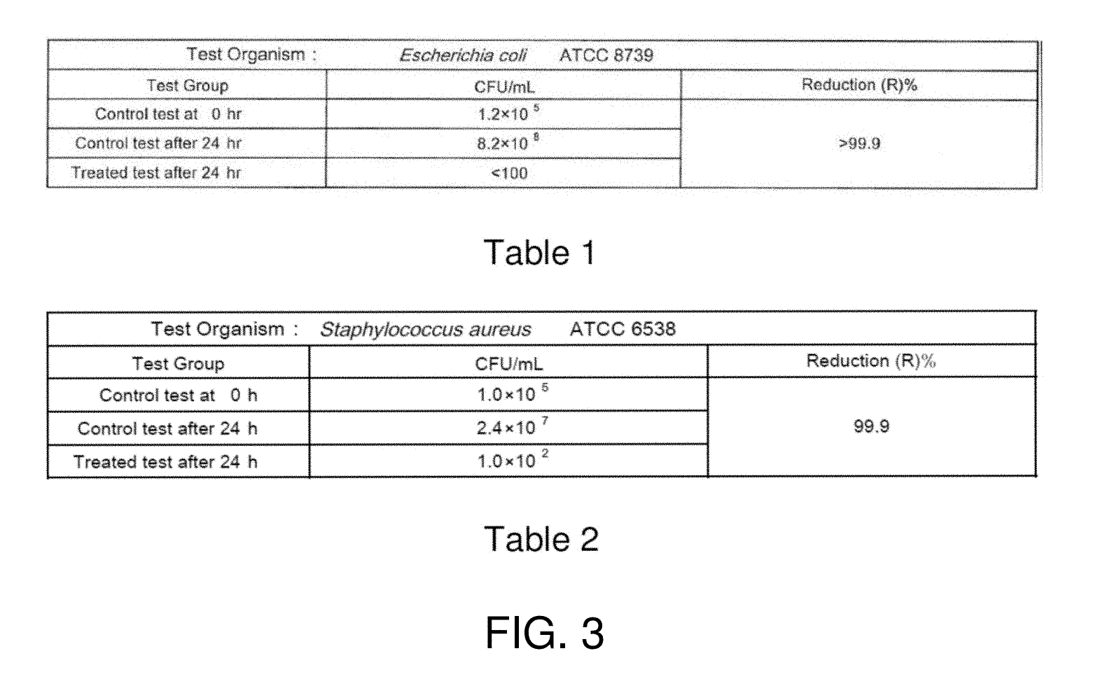

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of lighting apparatuses having different form factors.

The present disclosure discloses an air filtering apparatus comprising at least one air inflow port and at least one air outflow port, at least one forced air mechanism, and at least one air-permeable subsystem which further comprises at least three layers of air-filtering mesh structure, wherein at least one layer of air-filtering mesh structure contain aphotocatalytic-and-silver composite material for killing bacteria, and at least one layer of air-filtering mesh structure contains material for absorbing airborne non-microbial particles.

### Example Implementations

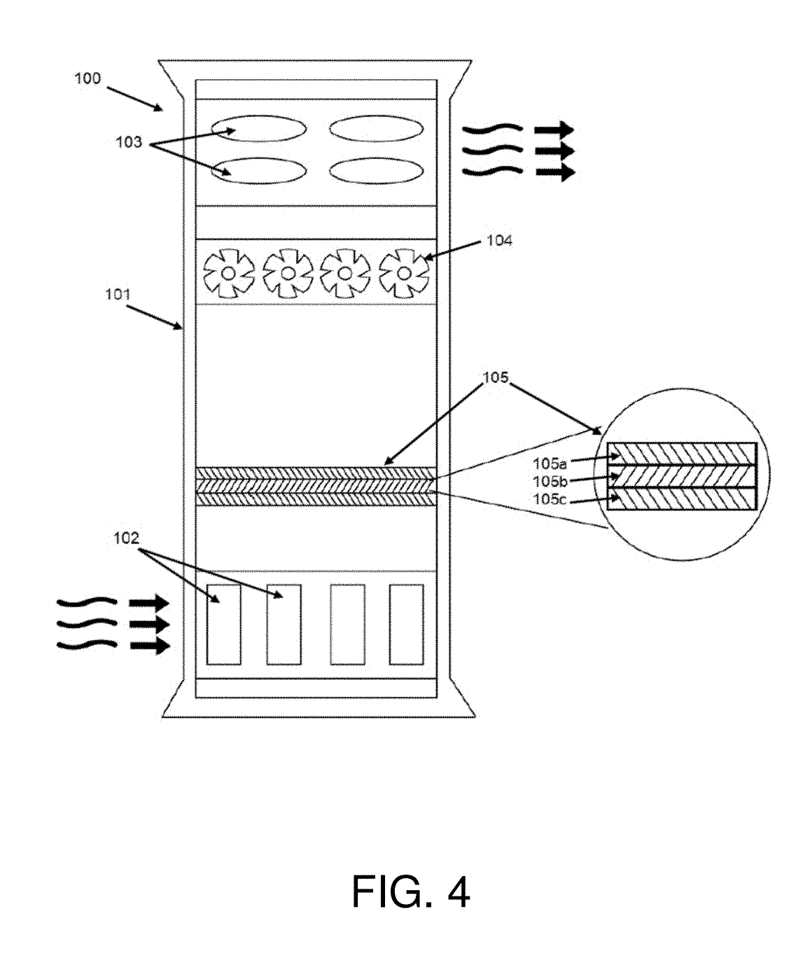

The FIG. 4 is an embodiment of the anti-bacterial air-filtering apparatus of the present disclosure in the form of a portable air-filtering machine 100. It has a housing 101, an air inflow port 102, an air outflow port 103, a fan 104 as the forced air mechanism, and a 3-layer air-filtering mesh structure 105. The fan 104 forces the air to flow from the air inflow port 102, through the 3-layer air filtering mesh structure 105, to the air outflow port 104. The two outer layers of the 3-layer air filtering mesh structure 105a and 105c are coated with a composite film of rhombus-shape anatase-type titanium oxide (TiO₂) and nano silver particles, and the center layer 105b contains activated carbon. The housing 101 is partially transparent so that ambient light can reach the composite film on the two outer layers 105a and 105c and activate the photocatalytic titanium oxide. This portable air-filtering machine 100 can be used in any indoor environment.

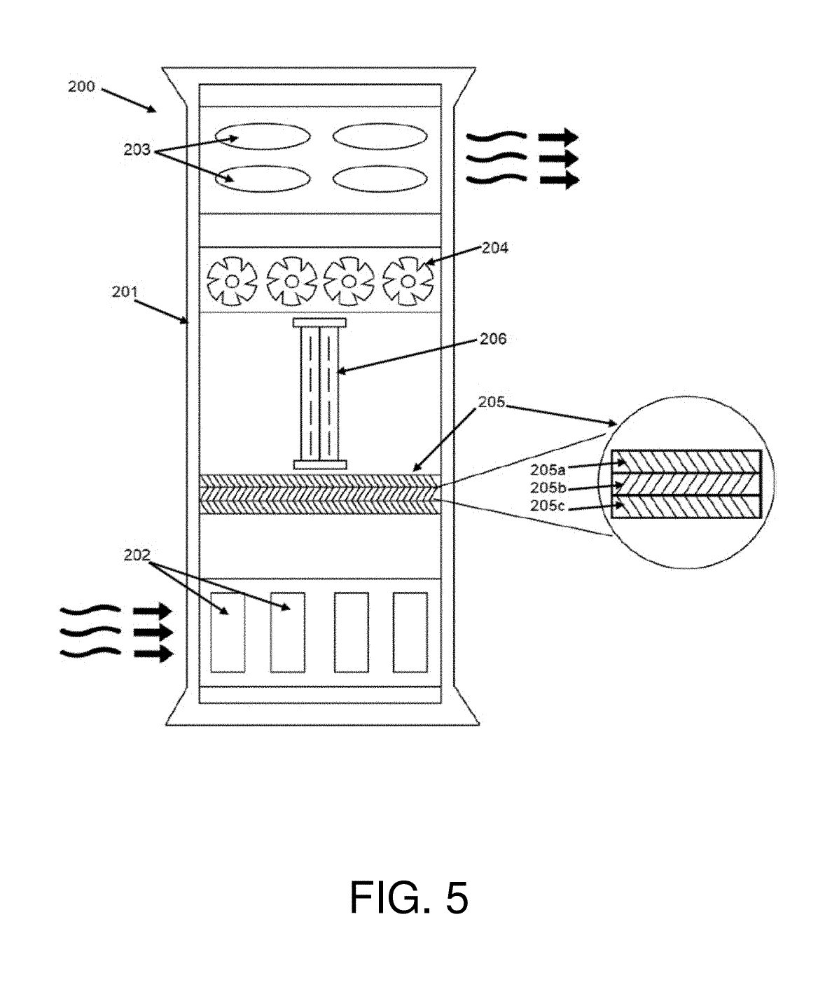

The FIG. 5 is an embodiment of the present disclosure. This portable air-filtering machine 200 is similar to the embodiment 100 in FIG. 4 with two distinctions. Firstly, it employs a LED light source 206, and secondly its housing 201 is opaque. Given the opaque housing 201, sufficient light may not penetrate the air inflow and outflow ports 202 and 203 and activate the photocatalytic titanium oxide on the two outer layers of the 3-layer air filtering mesh structure 205a and 205c. Therefore, a LED light source 206 is used inside the housing 201 for activating the photocatalytic titanium oxide. The LED light source is chosen for its long lifetime and a low efficacy depreciation where the typical LED light source's L70 maintenance hours are over 50,000 hours, i.e., the light source will depreciate less than 30% of its original light output over 50,000 hours of operation. With the close proximity of the LED light source 206 and the rhombus-shape anatase-type titanium oxide on two outer layers 205a and 205c (less than 6 inches), the photocatalytic bacteria-killing effect is guaranteed.

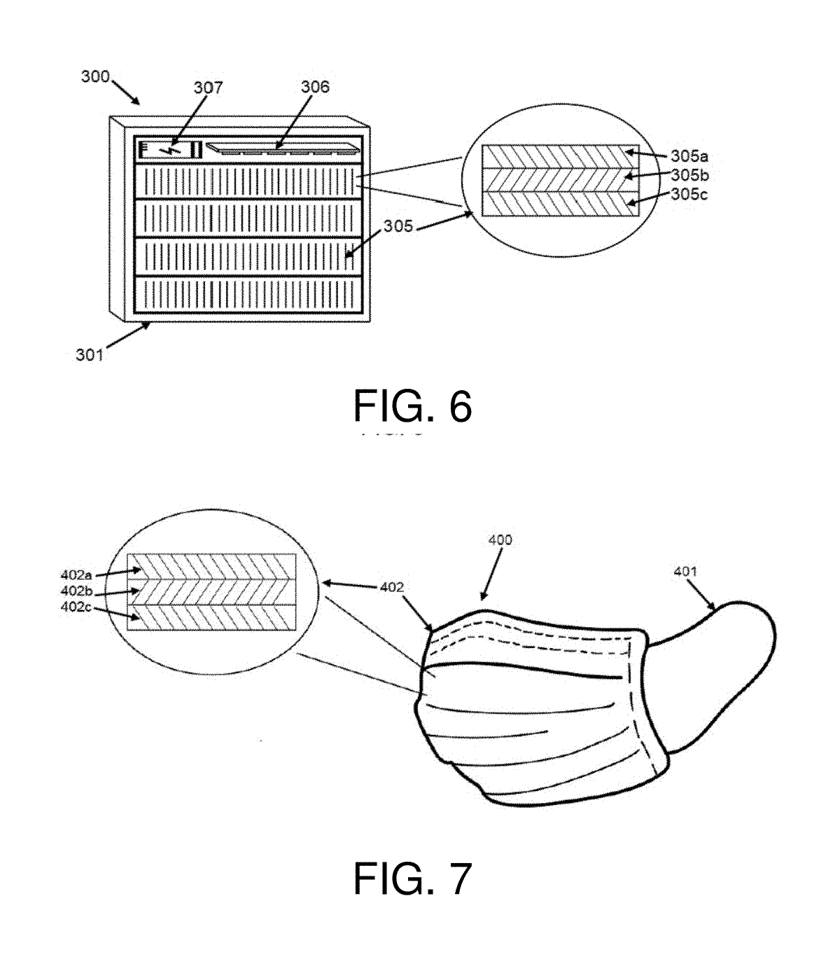

The FIG. 6 is an embodiment of the present disclosure in the form of an air-filter insert 300 which comprises an outer frame 301 around the edge, a 3-layer air filtering mesh structure 305, an LED strip light source 306, and a battery 307 for powering the LED light source. It is designed to be inserted in the airway of a HVAC. The forced air mechanism is part of the HVAC system and is external to the air-filter insert 300.The air is forced from the upstream airway 302 (i.e., the air inflow port) through the 3-layer air filtering mesh structure 305 to the downstream airway 303. The housing 301 of the HVAC airway is regarded as the housing of the present disclosure. The two outer layers of the 3-layer air filtering mesh structure 305a and 305c are coated with a composite film of rhombus-shape anatase-type titanium oxide (TiO₂) and nano silver particles, and the center layer 305b contains activated carbon. The battery-powered LED strip light source 306 activates the photocatalytic titanium oxide on the two outer layers 305a and 305c of the 3-layer air filtering mesh structure. The air-filter insert 300 may take a different, smaller form factor when designing for use in an automobile air circulation system.

The FIG. 7 is an embodiment of the present disclosure in the form of a face mask 400 which comprises a pair of elastic bands 401 for wearing the face mask over the ears and a 3-layer air filtering mesh structure 402. The two outer layers of the 3-layer air filtering mesh structure 402a and 402c are coated with a composite film of rhombus-shape anatase-type titanium oxide (TiO₂) and nano silver particles, and the center layer 402b contains electrostatic filter fabric.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. An anti-bacterial air filtering apparatus, comprising:
at least one light source;
a housing with at least one air inflow port and at least one air outflow port;
at least one forced air mechanism; and
at least one air-permeable subsystem,
wherein:
the forced air mechanism, disposed at least partially inside or outside of the housing, forces unfiltered air to flow into the apparatus through the at least one air inflow port and through the housing to provide filtered air out of the at least one air outflow port,
the air-permeable subsystem is disposed in an airway between the at least one air inflow port and the at least one air outflow port inside the house, and
the air-permeable subsystem comprises at least three layers of air-filtering mesh structure, wherein:
at least one layer of the air-filtering mesh structure contains an anti-bacterial composite material comprising photocatalytic particles and nano silver particles that filter microbial matters, and
at least one layer of the air-filtering mesh structure contains a material that absorbs airborne non-microbial particles.

2. The air-filtering apparatus of claim 1, wherein two layers of the air-filtering mesh structure contain an anti-bacterial composite material comprising photocatalytic particles and nano silver particles that filter microbial matters, wherein one layer of the air-filtering mesh structure contains a material that absorbs airborne non-microbial particles, and wherein two layers of anti-bacterial layers sandwich the material that absorbs non-microbial particle therebetween.

3. The air-filtering apparatus of claim 1, wherein the photocatalytic particles comprise rhombus-shape anatase-type titanium oxide (TiO₂).

4. The air-filtering apparatus of claim 1, wherein the material that absorbs airborne non-microbial particles comprises activated carbon.

5. The air-filtering apparatus of claim 1, wherein the material that absorbs airborne non-microbial particles comprises an electrostatic filter fabric.

6. The air-filtering apparatus of claim 1, wherein at least a part of the housing is transparent or translucent.

7. The air-filtering apparatus of claim 1, wherein the at least one light source is disposed inside the housing.

8. The air-filtering apparatus of claim 7, wherein a spectral power of ultraviolet (UV) components of the at least one light source is between zero and 5% of a total spectral power of the at least one light source.

9. The air-filtering apparatus of claim 7, wherein the at least one light source comprises a light emitting diode (LED).

10. The air-filtering apparatus of claim 9, wherein the LED is powered by a battery.

11. An anti-bacterial face mask device, comprising:
at least three layers of air-permeable mesh structure,
wherein two layers of the air-permeable mesh structure contain an anti-bacterial composite material comprising photocatalytic particles and nano silver particles that filter microbial matters,
wherein one layer of the air-permeable mesh structure contains a material that absorbs airborne non-microbial particles, and
wherein the two layers of the anti-bacterial layers sandwich the one layer that contains the material that absorbs airborne non-microbial particles therebetween.

12. The anti-bacterial face mask device of claim 11, wherein the photocatalytic particles comprise rhombus-shape anatase-type titanium oxide (TiO₂).

13. The anti-bacterial face mask device of claim 11, wherein the material that absorbs airborne non-microbial particles comprises activated carbon.

14. The anti-bacterial face mask device of claim 11, wherein the material that absorbs airborne non-microbial particles comprises an electrostatic filter fabric.
